(19) United States
(12) Patent Application Publication
(10) Pub. No.: US 2022/0196603 A1
(43) Pub. Date: Jun. 23, 2022

(54) MULTIMODE EXCITATION RESONANT GAS SENSOR AND METHOD

(71) Applicant: KING ABDULLAH UNIVERSITY OF SCIENCE AND TECHNOLOGY, Thuwal (SA)

(72) Inventors: Nizar Riyad Khaled JABER, Thuwal (SA); Amal HAJJAJ, Thuwal (SA); Nouha ALCHEIKH, Thuwal (SA); Mohammad Ibrahim YOUNIS, Thuwal (SA)

(21) Appl. No.: 17/441,939

(22) PCT Filed: Apr. 18, 2019
(86) PCT No.: PCT/IB2019/055844

§ 371 Date: Sep. 22, 2021

Related U.S. Application Data
(60) Provisional application No. 62/835,753, filed on Apr. 18, 2019.

(51) Int. Cl. G01N 29/036; G01N 33/00

(52) U.S. Cl. CPC G01N 29/036; G01N 2291/021; G01N 2291/014; G01N 33/0027

(57) ABSTRACT

A gas sensor includes a microbeam configured to vibrate when driven by a driving electrode; a vibrometer configured to measure a frequency associated with a vibration of the microbeam; a power source configured to apply a first voltage (V1) to the microbeam to heat the microbeam; and a controller configured to control the power source and to receive the frequency measured by the vibrometer. The controller controls the power source to heat the microbeam so that the microbeam is at a buckling point, and the controller determines at least one characteristic of a gas present around the microbeam, based on the frequency received from the vibrometer.

### CROSS-REFERENCE TO RELATED APPLICATIONS

This application claims priority to U.S. Provisional Patent Application No. 62/835,753, filed on Apr. 18, 2019, entitled “A RESONANT GAS SENSOR BASED ON MULTIMODE EXCITATION OF A BUCKLED BEAM,” the disclosure of which is incorporated herein by reference in its entirety.

### BACKGROUND

### Technical Field

Embodiments of the subject matter disclosed herein generally relate to a gas sensor, and more specifically, to a heated microbeam resonator that operates near a buckling point for detecting a gas based on multiple modes of vibration of the microbeam resonator.

### Discussion of the Background

Micro- and nano-electromechanical systems (MEMS and NEMS) have experienced rapid growth and demonstrated their potential in a wide range of sensing applications, such as ultra-small mass quantification, gas detection, pressure measurements, and charge sensing. Gas sensors that use M/NEMS structures have been employed for a wide range of applications, such as medical and healthcare applications, food and agriculture safety, and monitoring environmental conditions. This can be attributed to their small footprint and low power consumptions.

The quest for ultra-sensitive, low-cost, miniaturized gas sensors in the past few decades has sparked interest to seek techniques other than those used by the conventional gas sensors, which require large surface areas and special coating materials for selective and sensitive detection. Functionalization of the traditional gas sensor is realized by coating the surface of the MEMS gas sensor with a thin layer of a material that has a good affinity for the type of gas that is intended to be detected. For example, gold is used to detect mercury, a polymer doped with carbon nanotube is used to detect carbon dioxide, palladium is used for hydrogen sensing, and metal-organic frameworks are used for humidity and volatile organic compounds. The performance of such devices depends on the functionalization type, thickness of the beam, and the sensor design. The miniaturization of these devices is limited due to these factors.

MEMS gas sensors based on thermal conductivity measurements have been known to be among the promising alternative candidates. These sensors rely on the thermal energy dissipation (cooling or heating) of a heated structure due to the alteration of the surrounding gas concentration, which alleviates the requirement of large surface area and special techniques to functionalize the sensor. The temperature of the heated structure is altered by the surrounding gas properties, mainly due to the effective thermal conductivity of the ambient medium. These sensors are fabricated from materials with a high-temperature coefficient of resistance, which enables sensitive measurement of the bridge temperature by tracking the resistance value.

The micro-hotplates are the most developed and investigated MEMS structures used as thermal conductivity-based gas sensor. In recent studies, MEMS heated bridges have been employed to detect noble gases and they demonstrated promising results for binary gas mixtures. These sensors show long lifetime and great stability compared to the absorption-based gas sensors.

The thermal conductivity-based gas sensors rely on the resistance variation of the heated structures due to gas exposure, which is typically less than a few percent. In other words, a heated structure exchanges heat with the ambient atmosphere (which may be or not air) at a given rate. When the gas desired to be measured is present around the heated structure, the heat exchange between the heated structure and the new ambient changes, which impacts the resistivity of the heated structure. This change in resistivity is measured and mapped to the ambient gas. However, because the resistivity of the heated structure changes so little, this sensor is prone to errors. Thus, there is a strong demand for a more sensitive sensor to measure the ambient gas concentration.

Monitoring the frequency of MEMS resonators due to gas concentration change has demonstrated its applicability to the ultra-sensitive detection of gases up to the molecule levels [1, 2, 3, 4, 5]. Moreover, several dynamical features, such as the bifurcation points, internal resonance, and secondary resonances are utilized to improve the sensor sensitivity. In a previous work of the inventors [6], a buckling point (bifurcation point) of a heated silicon bridge was used to demonstrate the sensitivity and scalable nature of a pressure sensor. The concept is based on tracking the fundamental frequency of the electrothermally buckled bridge upon changing the surrounding pressure. Raising the surrounding pressure increases the heat dissipation, which reduces the bridge temperature. The cooling effect of the surrounding air changes the overall stress of the structure, and thus its fundamental frequency.

However, there is no known sensor available that is capable of measuring a gas presence and its concentration with high-accuracy. Thus, there is a need for a new gas sensor that can overcome the limitations discussed above.

### SUMMARY

According to an embodiment, there is a gas sensor that includes a microbeam configured to vibrate when driven by a driving electrode, a vibrometer configured to measure a frequency associated with a vibration of the microbeam, a power source configured to apply a first voltage (V1) to the microbeam to heat the microbeam, and a controller configured to control the power source and to receive the frequency measured by the vibrometer. The controller controls the power source to heat the microbeam so that the microbeam is at a buckling point, and the controller determines at least one characteristic of a gas present around the microbeam, based on the frequency received from the vibrometer.

According to another embodiment, there is a method for measuring a characteristic of a gas with a gas sensor, and the method includes vibrating a microbeam with a driving electrode, measuring with a vibrometer a frequency associated with a vibration of the microbeam, applying with a power source a first voltage (V1) to the microbeam to heat the microbeam, and determining with a controller, which is configured to control the power source and to receive the frequency measured by the vibrometer, the characteristic of the gas that is present around the microbeam, based on the frequency received from the vibrometer. The controller controls the power source to heat the microbeam so that the microbeam is at a buckling point.

According to still another embodiment, there is a gas sensor that includes a microbeam, a vibrometer configured to measure a frequency associated with a vibration of the microbeam, a power source that heats the microbeam, and a controller that controls the power source to heat the microbeam up to a buckling point, and determines a type of gas and a concentration of the gas present around the microbeam, based on the frequency measured by the vibrometer.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 is a schematic illustration of a novel gas sensor;

FIG. 2 illustrates a configuration that uses the novel gas sensor to determine the type of the gas and its concentration;

FIGS. 3A and 3B illustrate first and second vibration modes, respectively, of a microbeam of the gas sensor;

FIG. 4 illustrates the variation of the first and second frequencies (associated with the first and second modes, respectively) with the applied electrothermal voltage;

FIG. 5 illustrates the thermal conductivity of a number of gases used to test the gas sensor;

FIGS. 6A and 6B illustrate the relative change in the resistance of the gas sensor, over time, for various concentrations of the Nitrogen (N₂) mixed with Methane (CH₄) (A) and Carbon dioxide (CO₂) (B) for non-heated resonator for V_{Th}=10 mV;

FIGS. 7A and 7C illustrate the normalized resistance variation with time for different percentages of Nitrogen (N₂) mixed with (A) Methane (CH₄) and (C) Helium (He); FIGS. 7B and 7D illustrate the Normalized frequency shift with different percentages of Methane and Helium mixed with Nitrogen, respectively;

FIGS. 8A and 8C illustrate the normalized resistance variation with time for different percentages of Nitrogen (N₂) mixed with (A) Argon (Ar) and (C) Carbon Dioxide (CO₂); FIGS. 8B and 8D illustrate the Normalized frequency shift with different percentages of Argon and Carbon Dioxide mixed with Nitrogen, respectively;

FIG. 9 is a flowchart of a method for collecting data associated with the various gases to which the gas sensor is exposed to;

FIG. 10 is a flowchart of a method for determining a gas and its concentration with the novel gas sensor;

FIGS. 11A and 11B illustrate the response of the gas sensor when simulated for four different gases; and

FIG. 12 is a flowchart of another method for detecting a gas and its concentration with the gas sensor.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. For simplicity, the following embodiments are discussed with regard to a resonant gas sensor that includes a straight beam, which is clamped at both ends. However, the embodiments discussed herein are not limited to such a configuration, but they may be applied to other sensors and/or to a sensor that includes a beam that is not straight.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a new gas sensing technique is based on the simultaneous tracking of multiple modes of vibration of an electrothermally heated microbeam resonator operated near its buckling point. The new method maximizes the sensitivity of the sensor to the change in the gas concentration and enables the identification of the gas type without the need for selective coating. Under the same gas concentration value, the measurements demonstrate a 200% change in the frequency, in contrast to 0.5% when using the conventional technique based on resistance measurements. A resonant gas sensor that implements this novel method is also discussed. The resonant gas sensor is a low-cost gas sensor, and is capable of detecting a wide-range of gases as the present technique does not rely on surface functionalization. The novel resonant gas sensor is capable of not only measuring the gas concentration, but also identifying the gas type using an electrothermally heated microbeam operated near the buckling point. In one embodiment, the method simultaneously records frequencies associated with first and second vibrational modes of the microbeam while changing the gas concentration and type.

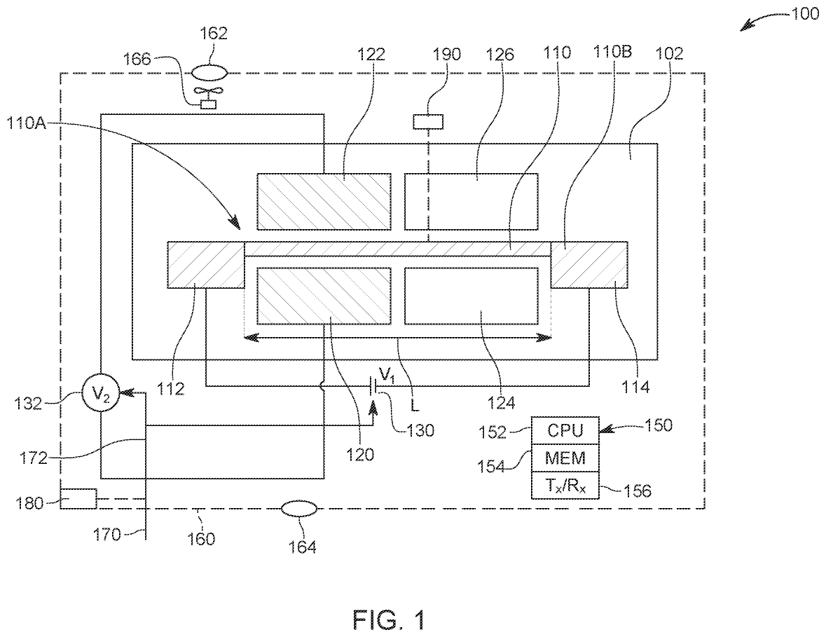

The method and new sensor are now discussed in more detail with regard to the figures. FIG. 1 shows a gas sensor 100 having an in-plane, clamped-clamped, microbeam 110 that has its two ends 110A and 1108 fixed (clamped) to anchors/pads 112 and 114, respectively. A driving electrode 120 and a sensing electrode 122 are formed to sandwich the microbeam 110. Note that the middle region of the microbeam 110 is not attached to a substrate 102, which supports all these elements, so that the microbeam can move/oscillate relative to the substrate while its ends 110A and 1108 are fixed relative to the substrate.

The microbeam 110 is fabricated, in one embodiment, from a highly conductive 30 μm thick silicon layer using, for example, a surface micromachining process. This process may include the steps of lithography to transfer the device design from the mask to the wafer, metal sputtering to create the connection pads, deep reactive ion etch (DRIE) to form the structure, and vapor hydrofluoric acid dry etch (vpour-HF) to release the device. In this embodiment, the microbeam 110 has a length L of 500 μm, width of 30 μm, and a thickness of 2 μm. Those skilled in the art would understand that other values may be used as long as these dimensions remain in the order of pm, i.e., less than 1 mm.

The microbeam 110 may be separated from the driving electrode 120 with a transduction gap of 8 μm. In this embodiment, a half electrode configuration is used to break the symmetry of the excitation force, which enables the excitation of the antisymmetric modes. This means that the driving electrode 120 and the sensing electrode 122 do not extend over the entire length L of the microelectrode 110, but only about half of this length. Thus, electrodes 124 and 126, if present, are not electrically connected to any device. FIG. 1 shows that the pads 112 and 114 are electrically connected to a first power source 130, that generates a first voltage V1, and the driving electrode 120 and the sensing electrode 122 are electrically connected to a second power source 132, that generates a second voltage V2. Each of these two sources can be a direct current (DC) or alternative current (AC) power source. In the embodiment of FIG. 1, the first power source 130 is a DC power source and the second power source 132 is an AC power source. In one embodiment, the power sources 130 and 132 may be implemented into a single physical power source.

The gas sensor 100 may also include a controller 150 that includes at least a processor 152 and a memory 154. The processor may be connected in a wired or wireless manner to the voltage sources 130 and 132 to control the voltages applied to the microbeam 110 and the driving electrode 120, respectively. The controller 150 may also include a transceiver unit 156 for communicating in a wireless manner (e.g., radio-frequency or Wi-Fi) with a remote global controller to alert the operator of the gas sensor about a possible detection of the target gas. All these elements may be then placed in a housing 160 to protect them from adverse conditions. The housing 160 may have at least an inlet port 162 and an outlet port 164 to allow the ambient atmosphere to enter inside the housing and interact with the microbeam. In one application, a fan 166 or similar device may be placed on the housing, to force the ambient air to enter inside the housing. The power necessary for the gas sensor 100 may be supplied from the grid 170 through a cable 172 or from battery, solar cell, wind turbine, fuel cell 180. A vibrometer device 190 may also be placed inside the housing 160 for monitoring and measuring a vibration of the microbeam 110. The vibrometer 190 may be controlled by the controller 150 and may be supplied with power from the grid 170 or from the alternative supply 180 or both.

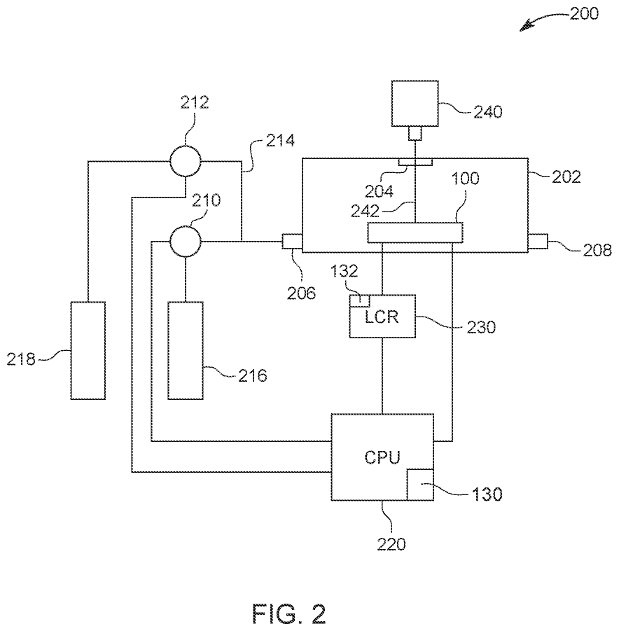

For experimentally determining the concentration and type of the gas surrounding the microbeam of the gas sensor, the gas sensor 100 (the microbeam 110 and its associated electrodes) is placed in a chamber 202 of a gas sensor system 200 as illustrated in FIG. 2. Chamber 202 can be made of any material as long as there is a window 204 that is transparent to a laser light, to be discussed later. Chamber 202 may have an input port 206 and an output port 208. Except for the input and output shown in FIG. 2, chamber 202 is sealed from the ambient. The input port 206 is configured to allow a controlled amount of gas to enter the chamber and the output port 208 is configured to allow the gas from the chamber to be discharged into the environment. As the output port 208 communicates freely with the ambient, the measurements to be discussed next are performed at atmospheric pressure and room temperature. The chamber is used to control the amount of gas that is provided around the microbeam 110 of the gas sensor 100 for experimental measurement purposes within a lab. However, as discussed later, if the gas sensor 100 is incorporated into a gas sensor system for use in the field, the structure shown in FIG. 1 may be used.

The input port 206 is fluidly connected in this experimental setup to two mass flow controllers 210 and 212, through piping 214. The mass flow controllers are configured to control a mass amount of a gas that is provided to the input port 206, from corresponding gas storage containers 216 and 218, respectively. For example, in one embodiment, the first gas storage container 216 holds dry nitrogen (N2) while the second gas storage container 218 holds a targeted gas (e.g., helium, methane, argon, carbon dioxide, etc.). The mass flow controllers 210 and 212 may be connected to a controller 220 for automatically controlling an amount of the gas that is released into the chamber 202. Controller 220 may be a computing system, a computer, etc. that includes at least a processor. Controller 220 may also host the first source 130 discussed with regard to FIG. 1.

Controller 220 is electrically connected to the gas sensor 100 for supplying the first voltage V1. The first voltage V1 is applied to the pads 112 and 114 of the microbeam 110 for controlling a temperature of the microbeam. Note that by applying the first voltage V1 to the microbeam, a temperature of the beam may be increasing. FIG. 2 also shows a measurement device 230 that is capable of measuring a resistance, impedance and/or capacitance of the gas sensor 100. The measurement device 230 may include the second source 132. The measurement device 230 is connected to the driving electrode 120 and the sensing electrode 122 for driving the microbeam 110 with a desired frequency. In one embodiment, the measurement device 230 may be combined with the controller 220 to have a single device that performs the functions of the measurement device and the controlling device.

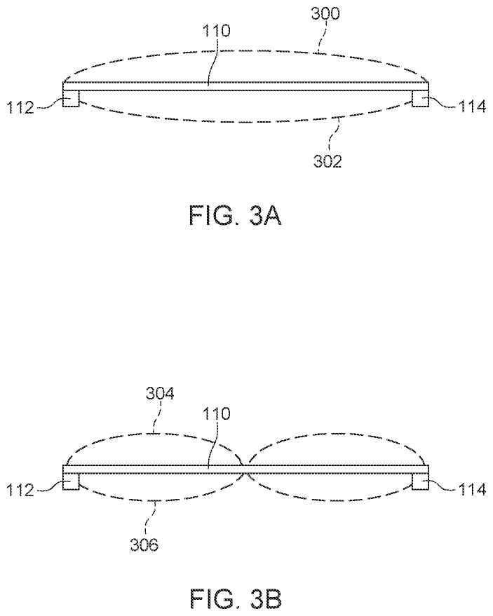

If the measurement device applies a certain voltage V2 to the electrodes sandwiching the microbeam 110, it is possible to make the microbeam to oscillate/vibrate at its fundamental (or resonance) frequency f1, as illustrated in FIG. 3A, which is also called 1^{st} mode of vibration. Note that in FIG. 3A, the microbeam 110 oscillates between positions 300 and 302 during the 1^{st} mode. For a different voltage V2′ applied to the electrodes sandwiching the microbeam 110, it is possible to make the microbeam to oscillate with a first harmonic f2 of the fundamental frequency, as illustrated in FIG. 3B, which is also called the 2^{nd} mode of vibration. In FIG. 3B, the microbeam 110 oscillates between positions 304 and 306.

To detect these frequencies or modes, the vibrometer device 240 may be used for measuring the vibrations of the microbeam 110. In one embodiment, the vibrometer device is a laser Doppler vibrometer. Other devices may be used for measuring the vibrations of the microbeam 110. The vibrometer 240 sends a light beam 242 through the window 204 to impinge on a surface of the microbeam 110, as illustrated in FIG. 2. Note that window 204 may be made of any material that allows a light or laser beam to pass through it.

When in operation, the gas sensor system 200 applies the first voltage V1 to the pads 112 and 114. Due to the current passing through the microbeam 110, heat is generated in the microbeam and the length L of the microbeam starts to increase. However, because the ends 110A and 1108 of the microbeam 110 are fixedly attached to the pads 112 and 114, respectively, the microbeam cannot expand. Thus, when a certain amount of heat is generated by the current flowing along the microbeam, the microbeam will buckle, i.e., it will bend somewhere between the ends 110A and 1108 to allow for further expansion of the microbeam. This is called the buckling effect and it happens at a given buckling voltage. Note that by heating the microbeam, the frequencies of the first and second modes also change.

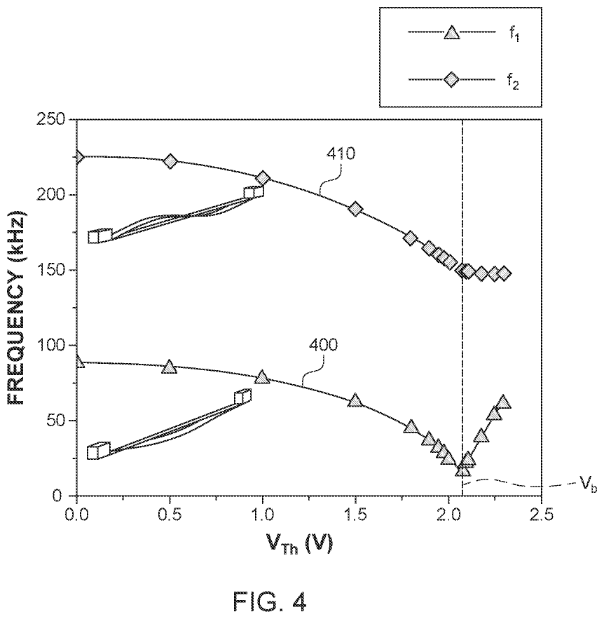

In this regard, FIG. 4 shows the frequencies f1 and f2 associated with the first mode 400 and the second mode 410 as a function of the applied voltage V1 to the pads 112 and 114. It is noted that both frequencies f1 and f2 decrease when the voltage increases, up to the buckling voltage Vb. At the buckling voltage Vb, the first mode frequency f1 experiences a change in its change, i.e., the first mode frequency increases as the voltage increases. The second mode frequency f2 still decreases as the voltage increases past the buckling point, but at a smaller rate than prior to reaching the buckling voltage, as also shown in FIG. 4.

The decrease of the first mode frequency can be attributed to the decrease in the microbeam stiffness due to the induced compressive stress until the buckling point. After buckling, a sudden increase in the first mode (first symmetric mode) is observed due to the stretching mechanism, which increases the stiffness of the buckled microbeam. The second mode (first antisymmetric mode) frequency remains constant and is not affected with the stretching because of the pure bending nature of the second mode shape. Operating the microbeam resonator near the buckling point maximizes the sensitivity of the gas sensor to a variation in the first mode frequency due to physical phenomena that affect the axial stress such as cooling and heating effects. Exposing the microbeam to gases with higher/lower thermal conductivity then Nitrogen reduces/increases the axial stress inside the microbeam, which alters the values of the resonance frequencies. Hence, by tracking the shift in frequency associated with the first mode, it is possible to quantify the concentration of the targeted gas, while by tracking the shift in the frequency associated with the second mode, it is possible to identify the type of gas (having higher/lower thermal conductivity compared to Nitrogen).

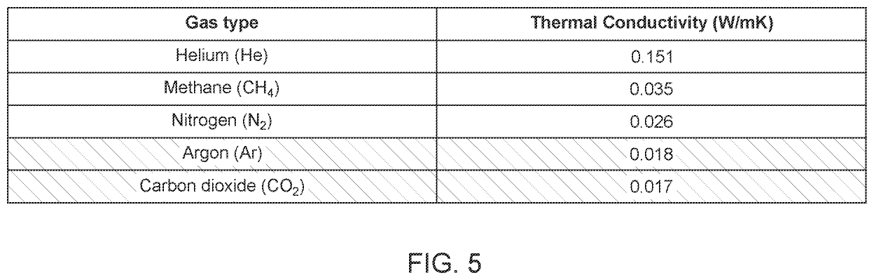

To exemplify the above noted operation principle, the inventors have exposed the microbeam 110 to gases (e.g., Helium (He) and Methane (CH4)) which have a higher thermal conductivity than the Nitrogen and also to gases (e.g., Carbon dioxide (CO2) and Argon (Ar)) which have a lower thermal conductivity compared with Nitrogen, as illustrated in Table 1 in FIG. 5.

A real-time measurement of the resistance variation of the microbeam 110 was performed for different gas concentrations in order to check the repeatability and the time response to the gas exposure. For a doped Silicon microbeam resonator, its resistance variation with the temperature is assumed to be linear, and hence the variation of the resistance at different gas concentrations can be correlated to the temperature variation of the microbeam. The resistance variation is correlated to the stress variation, and hence the resonance frequency variation. Tracking the resistance variation to different gas concentrations of a heated microbeam resonator has been used to prove the reversibility and the repeatability of the proposed technique.

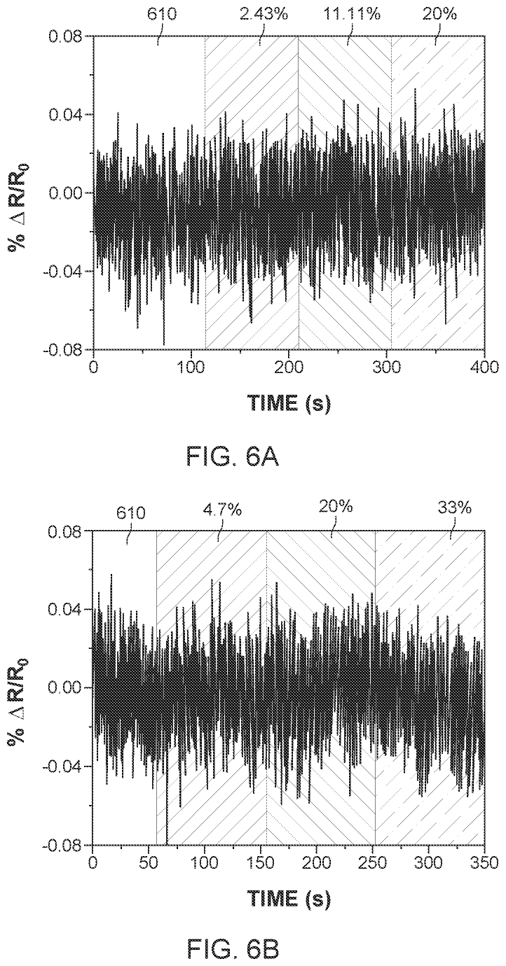

Thus, in a first experiment, the chamber 202 was flushed with Nitrogen at atmospheric pressure and ambient temperature. At almost zero electrothermal voltage (10 mV) V1, the resistance variation ΔR/R of the microbeam 110 was measured at different concentrations of Methane mixed with Nitrogen and Carbon dioxide mixed with Nitrogen. Note that the Methane and Carbon dioxide have higher and lower thermal conductivities compared with Nitrogen, respectively. In this respect, FIGS. 6A and 6B show that no effective resistance variation is reported when varying the gas concentration for each gas. Note that R₀ indicates the mean resistance of the microbeam when flushed with Nitrogen. Zones 610 indicate the Nitrogen flushing of the chamber for each gas. This indicates that there is no physical or chemical interaction between the microbeam and the gas molecules in the chamber.

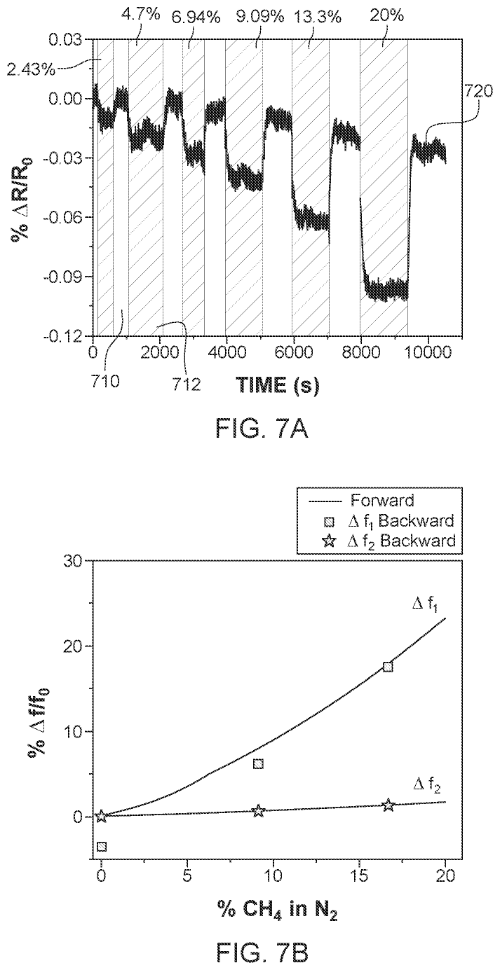

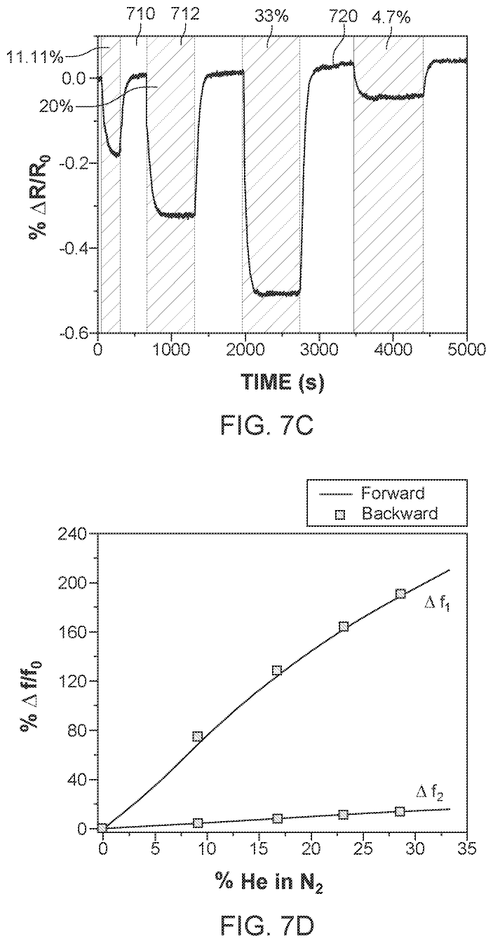

Next, the system 200 was used to tune the electrothermal voltage V1 to bring the microbeam 110 at the buckling point for all the gas measurements, to maximize the sensor's sensitivity to any axial change due to the presence of different gases. Firstly, the chamber 202 is exposed to Methane mixed with Nitrogen and having different concentrations of Methane, without exceeding 20% for safety reason, while recording the real-time variation of the resistance of the microbeam, as illustrated in FIG. 7A. Zones 710 in FIG. 7A indicate the Nitrogen flushing of the chamber 202. FIG. 7A shows that as the concentration of Methane increases (see zones 712), the resistance of the microbeam decreases due to the cooling of the microbeam promoted by the Methane. This proves that as the Methane concentration increases, the effective thermal conductivity of the medium surrounding the microbeam increases, thus leading to more convection of the microbeam surface, which reduces the microbeam temperature. The reduction in the temperature reduces the compressive force, which increases the first and second resonance frequencies as shown in FIG. 7B. The normalized frequency shift is illustrated in FIG. 7B when increasing (forward) and decreasing (backward) the Methane concentration in the chamber. The first voltage V1 is fixed for all the results at the buckling voltage to maximize the sensor's sensitivity.

Next, the microbeam 110 was exposed to a mixture of Helium and Nitrogen, where the Helium has a higher thermal conductivity compared to the Nitrogen. FIG. 7C shows a decrease in the resistance of the microbeam with the increased of the Helium concentration. Also, the normalized frequency shift is demonstrated to be higher than for Methane. As shown in FIG. 7D, exposing the microbeam 110 to different concentrations of Helium increases the frequency values of the first and second modes due to the increase in the effective thermal conductivity that cools down the microbeam, and hence decreases the compressive axial stress of the microbeam. A very high-sensitivity is shown for the same concentration of Helium, reaching 200% in FIG. 7D, when compared to the Methane case, which reaches 25% in FIG. 7B. This large difference could be explained by the higher thermal conductivity of Helium comparing to Methane (4 times higher as illustrated in Table 1 in FIG. 5). As shown in FIGS. 7A and 7C, the response 720 of the microbeam 110 returns to the original value upon flushing the chamber with nitrogen, which confirms the reversibility of the gas sensor.

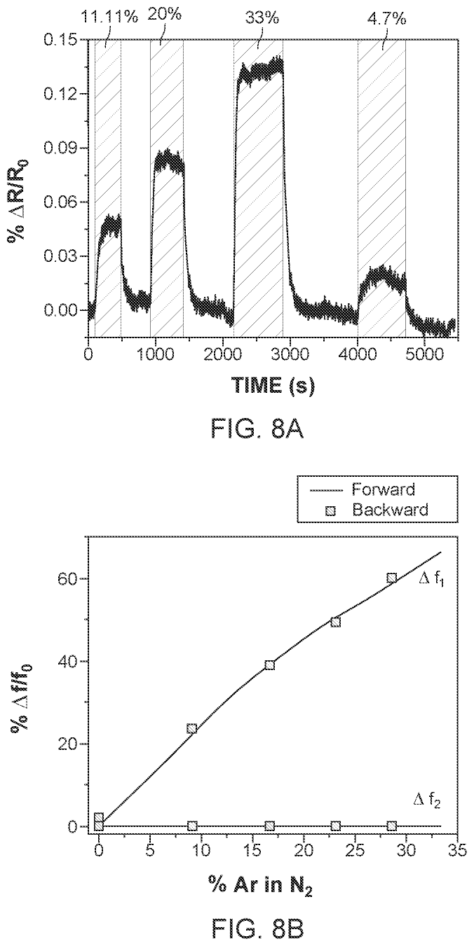

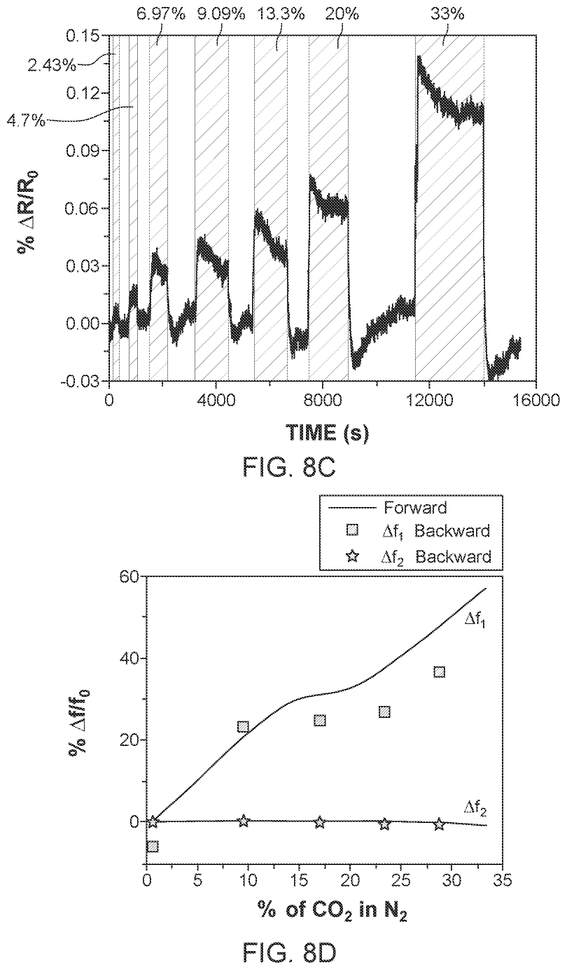

On the other hand, exposing the microbeam resonator to a mixture of Argon and Nitrogen increases the resistance of the microbeam 110, as depicted in FIG. 8A. Increasing the Argon concentration decreases the effective thermal conductivity of the medium surrounding the microbeam; hence, less heat convection takes place from the microbeam. This increases the temperature of the microbeam, and thus, the compressive stress in the microbeam. Increasing the Argon concentration raises the frequency of the first mode while the second mode remains constant, as illustrated in FIG. 8B. Similar results are obtained for a mixture of Carbon dioxide and Nitrogen in FIGS. 8C and 8D. The same percentage of the frequency variation of the first resonance frequency was shown for both Argon and Carbon dioxide, reaching 60%, since they have almost the same value of thermal conductivity.

The fact that each tested gas has its own unique signature in terms of increasing or decreasing the resistance of the microbeam, and also in terms of the behavior of the first and second mode frequencies around the buckling point, makes it possible to experimentally map each gas that can be found in the atmosphere or is of interest to these characteristics, to store these characteristics into the memory that is associated with the controller of the gas sensor, and then to use the gas sensor in any desired environment for detecting the presence of a certain gas and its concentration.

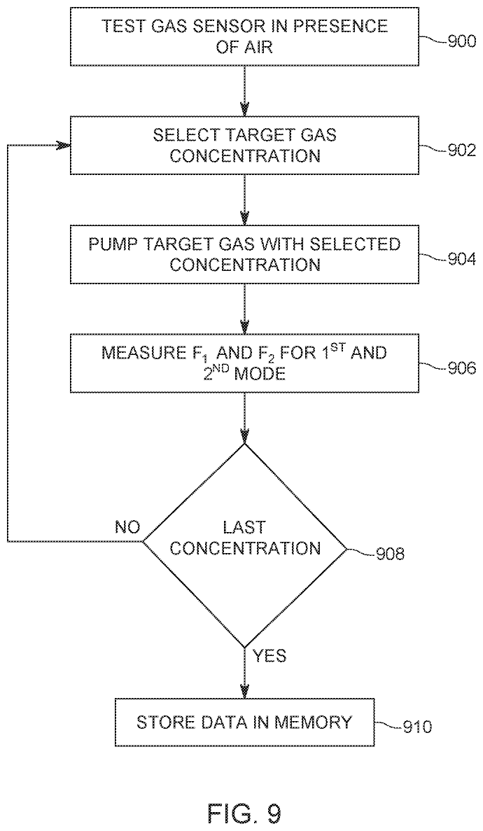

In this respect, it is possible to build a purpose-oriented sensor and to configure it to detect one or more specific gases. For example, suppose that in a given environment (e.g., a mine, an industrial facility, a metro station, a nuclear power station, or any desired facility), it is desired to be able to monitor whether methane is present and its concentration as this gas is dangerous to humans when present over a certain concentration. If this is the purpose of the gas sensor, then the gas sensor is tested in step 900 (as illustrated in the flowchart of FIG. 9) in the lab, in the presence of ambient air, to establish a background for the first and second mode frequencies of microbeam 110. Note that the voltage V1 applied to the pads of the microbeam 110 is selected so that the microbeam is at its buckling point (or voltage). Then, in step 902, a first concentration of the methane is selected and the methane with this concentration is pumped in step 904 around the microbeam. The methane is pumped mixed with air to have the first concentration. The first and second mode frequencies of the microbeam in this new environment are measured in step 906. In step 908, an evaluation is made of whether a new concentration of methane should be tested. If the answer is yes, the process returns to step 902. Note that the number of methane concentrations is selected by the operator of the gas sensor and reflects the desired accuracy that the gas sensor should have. If the answer is no in step 908, all the collected data is stored in a memory in step 910, which is associated with the gas sensor.

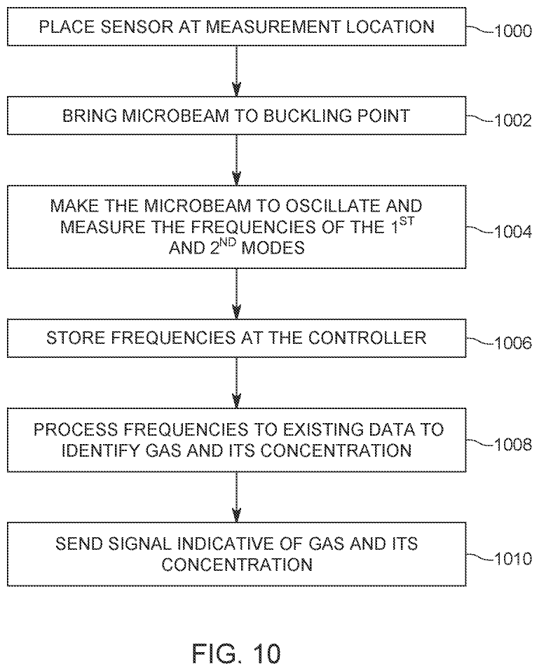

Later, when the gas sensor is deployed for actual measurements, the stored data is used to identify the gas and the concentration of the gas from the actual readings of the gas sensor as discussed with now with regard to FIG. 10. In step 1000, the gas sensor 100 (illustrated in FIG. 1) is placed at the desired location to be monitored. In step 1002, the gas sensor 100 is activated, i.e., the first voltage V1 is applied to heat the microbeam 110 so that the gas sensor is at the buckling point. In step 1004, the controller 150 applies the second voltage V2 and then measures the frequencies f1 and f2 of the first and second modes of the microbeam with a vibrometer 190. In step 1006, the measured frequencies are received at the controller 150 and stored in the memory 154. In step 1008, the processor 152 compares the received frequencies with those stored in the memory 154 (see method of FIG. 9 for generating this data). The processor 152 then identifies the detected gas and its concentration by mapping (1) the measured frequencies for the first and second modes to (2) the already recorded frequencies of the various gasses previously tested. While it is possible to test a single gas in the method illustrated in FIG. 9, one skilled in the art would understand that the same method may be used to test many gases and associated concentrations and to store all this data to create a library of first and second mode frequencies for the tested gasses. As previously discussed, from the trend of the second frequency of the first mode, it is possible to identify the gas, and then from the first frequency of the second mode, it is possible to determine the concentration of the target gas.

The gas sensor 100 has been tested by simulation under the following conditions. Various solvers have been coupled including the Solid Mechanics, Electric Currents, and Heat Transfer interface domains. A model couples the Joules heating of the doped polysilicon microbeam with the convective heat transfer for the surrounding gas. The anchors 112 and 114 of the microbeam 110 are assigned a fixed constraint boundary condition with ambient temperature at their bottom. The rest of the faces of the structure are set to a convective heat boundary condition, where the heat flux option is used for external natural convection with the gas mixture (the mixture of Nitrogen and the targeted gas) at ambient temperature and atmospheric pressure. The model takes into account the temperature dependence of the thermal properties of the gas mixture and the polysilicon microbeam. The effective thermal conductivity of the gas mixture, known to be a nonlinear function of different parameters (thermal conductivity, viscosity, molar mass of each gas), was calculated using the Wassiljewa formula and then implemented in the software.

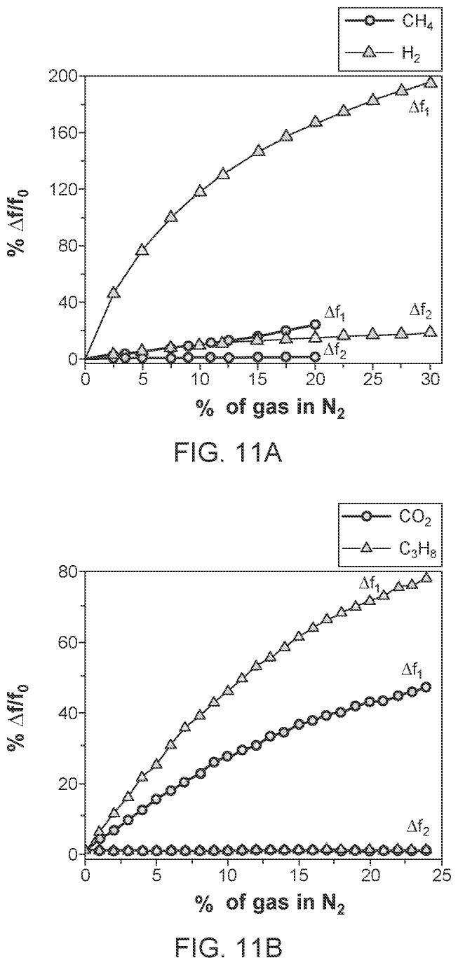

The simulated results of the resonance frequencies variation of the microbeam 110 with different gas concentrations for Hydrogen, Methane, Propane, and Carbon dioxide are illustrated in FIGS. 11A and 11B. FIGS. 11A and 11B show simulated normalized frequency shifts with different percentages of various gases mixed with Nitrogen. The simulation results prove that each gas has a unique signature when considering the first and second frequencies associated with the first and second modes of the microbeam, and these signatures remain unique for the various concentrations of these gases. An advantage of this gas sensor is the capacity to detect different harmful gases without the need for any specific functionalization of the microbeam for each gas.

The novel gas sensor 100 introduced above is configured for measuring the gas concentration and identifying the gas type using an electrothermally heated microbeam, which is operated near the buckling point. The method for identifying the gas and measuring its concentration is based on the simultaneous tracking of the first and second modes at different gas concentration levels. The frequency values depend on the effective convective cooling/heating of the gas mixture surrounding the heated microbeam (i.e., MEMS resonator). By tracking the shift in the first mode, the concentration of the targeted gas could be quantified. The shift in the second mode is employed to determine the type of gas (having higher/lower effective thermal conductivity compared to Nitrogen). The results demonstrate the possibility of realizing a gas sensor that can determine the type of gas without the need for selective coating. In contrast to resistance-based measurements, operating the microbeam near the buckling point and monitoring the frequency shift maximize the sensor sensitivity. Under the same value of gas concentration, the results show a 200% relative frequency change compared to a few percent when tracking the resistance value of an existing gas sensor. An advantage of the proposed scalable gas sensor is the simplicity of fabrication, operating principle, and sensing scheme. Also, the proposed gas sensor is targeting a wide range of gases as the presented technique does not rely on surface functionalization.

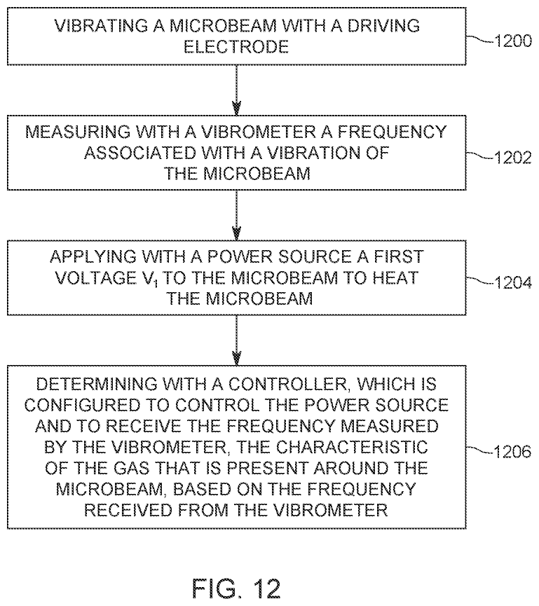

A method for measuring a characteristic of a gas with a gas sensor as illustrated in FIG. 1 is now discussed with regard to FIG. 12. The method includes a step 1200 of vibrating a microbeam 110 with a driving electrode 120, a step 1202 of measuring with a vibrometer 190 a frequency associated with a vibration of the microbeam 110, a step 1204 of applying with a power source 130, 132 a first voltage V1 to the microbeam 110 to heat the microbeam 110, and a step 1206 of determining with a controller 150, which is configured to control the power source 130, 132, and to receive the frequency measured by the vibrometer 190, the characteristic of the gas that is present around the microbeam 110, based on the frequency received from the vibrometer 190. The controller controls the power source to heat the microbeam so that the microbeam is at a buckling point.

In one application, the characteristic includes a type of gas and a concentration of the gas. The buckling point corresponds to when the microbeam changes its shape from its original shape to a buckled line. The method may further include a step of applying with a first source a first voltage V1 for heating the microbeam, and a step of applying with a second source a driving signal to the driving electrode to make the microbeam to vibrate. The frequency includes a first frequency corresponding to a first mode of vibration of the microbeam and a second frequency corresponding to a second mode of vibration of the microbeam. The first mode of vibration corresponds to a resonance of the microbeam and the second mode of vibration corresponds to a first harmonic of the resonance. In one application, the vibrometer is a laser Doppler vibrometer. In one embodiment, the microbeam has a length smaller than 1 mm.

The disclosed embodiments provide a novel gas sensor and method for determining the presence of a gas and its concentration by measuring frequencies of a microbeam that is at a buckling voltage. The embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] N. Jaber, S. Ilyas, O. Shekhah, M. Eddaoudi, and M. Younis, “Multimode excitation of a metal organics frameworks coated microbeam for smart gas sensing and actuation,” Sensors and Actuators A: Physical, vol. 283, pp. 254-262, 2018.
[2] T. Thundat, E. Wachter, S. Sharp, and R. Warmack, “Detection of mercury vapor using resonating microcantilevers,” Applied Physics Letters, vol. 66, pp. 1695-1697, 1995.
[3] J. Henriksson, L. G. Villanueva, and J. Brugger, “Ultra-low power hydrogen sensing based on a palladium-coated nanomechanical beam resonator,” Nanoscale, vol. 4, pp. 5059-5064, 2012.
[4] N. Jaber, S. Ilyas, O. Shekhah, M. Eddaoudi, and M. I. Younis, “Multimode MEMS Resonator for Simultaneous Sensing of Vapor Concentration and Temperature,” IEEE Sensors Journal, vol. 18, pp. 10145-10153, 2018.
[5] Y. Hwang, H. Sohn, A. Phan, O. M. Yaghi, and R. N. Candler, “Dielectrophoresis-assembled zeolitic imidazolate framework nanoparticle-coupled resonators for highly sensitive and selective gas detection,” Nano letters, vol. 13, pp. 5271-5276, 2013.
[6] A. Z. Hajjaj, N. Alcheikh, M. A. A. Hafiz, S. Ilyas, and M. I. Younis, “A scalable pressure sensor based on an electrothermally and electrostatically operated resonator,” Applied Physics Letters, vol. 111, p. 223503, 2017.

1. A gas sensor comprising:
a microbeam configured to vibrate when driven by a driving electrode;
a vibrometer configured to measure a frequency associated with a vibration of the microbeam;
a power source configured to apply a first voltage (V1) to the microbeam to heat the microbeam; and
a controller configured to control the power source and to receive the frequency measured by the vibrometer,
wherein the controller controls the power source to heat the microbeam so that the microbeam is at a buckling point, and
wherein the controller determines at least one characteristic of a gas present around the microbeam, based on the frequency received from the vibrometer.

2. The gas sensor of claim 1, wherein the at least one characteristic includes a type of gas and a concentration of the gas.

3. The gas sensor of claim 1, wherein the buckling point corresponds to when the microbeam changes its shape from an original shape to a buckled line.

4. The gas sensor of claim 1, wherein the power source includes a first source that applies the first voltage V1 for heating the microbeam and a second source that applies a driving voltage to the driving electrode to make the microbeam to vibrate.

5. The gas sensor of claim 1, wherein the frequency includes a first frequency corresponding to a first mode of vibration of the microbeam and a second frequency corresponding to a second mode of vibration of the microbeam.

6. The gas sensor of claim 5, wherein the first mode of vibration corresponds to a resonance of the microbeam and the second mode of vibration corresponds to a first harmonic of the resonance.

7. The gas sensor of claim 1, wherein the vibrometer is a laser Doppler vibrometer.

8. The gas sensor of claim 1, wherein the microbeam has a length smaller than 1 mm.

9. The gas sensor of claim 1, wherein the microbeam has a length of about 0.5 mm.

10. The gas sensor of claim 1, wherein the microbeam is clamped at each end.

11. The gas sensor of claim 1, further comprising:
a driving electrode connected to the power source and configured to make the microbeam vibrate,
wherein the driving electrode is half a length of the microbeam.

12. A method for measuring a characteristic of a gas with a gas sensor, the method comprising:
vibrating a microbeam with a driving electrode;
measuring with a vibrometer a frequency associated with a vibration of the microbeam;
applying with a power source a first voltage (V1) to the microbeam to heat the microbeam; and
determining with a controller, which is configured to control the power source and to receive the frequency measured by the vibrometer, the characteristic of the gas that is present around the microbeam, based on the frequency received from the vibrometer,
wherein the controller controls the power source to heat the microbeam so that the microbeam is at a buckling point.

13. The method of claim 12, wherein the characteristic includes a type of gas and a concentration of the gas.

14. The method of claim 12, wherein the buckling point corresponds to when the microbeam changes its shape from an original shape to a buckled line.

15. The method of claim 12, further comprising:
applying with a first source the first voltage V1 for heating the microbeam; and
applying with a second source a driving voltage to the driving electrode to make the microbeam to vibrate.

16. The method of claim 12, wherein the frequency includes a first frequency corresponding to a first mode of vibration of the microbeam and a second frequency corresponding to a second mode of vibration of the microbeam.

17. The method of claim 16, wherein the first mode of vibration corresponds to a resonance of the microbeam and the second mode of vibration corresponds to a first harmonic of the resonance.

18. The method of claim 12, wherein the vibrometer is a laser Doppler vibrometer.

19. The method of claim 12, wherein the microbeam has a length smaller than 1 mm.

20. A gas sensor comprising:
a microbeam;
a vibrometer configured to measure a frequency associated with a vibration of the microbeam;
a power source that heats the microbeam; and
a controller that controls the power source to heat the microbeam up to a buckling point, and determines a type of gas and a concentration of the gas present around the microbeam, based on the frequency measured by the vibrometer.
